(19) United States
(12) Patent Application Publication
(10) Pub. No.: US 2017/0209483 A1
(43) Pub. Date: Jul. 27, 2017

(54) ANTIBIOTIC ACTIVITY OF IRON SEQUESTERING POLYMERS

(71) Applicant: UNIVERSITY OF KANSAS, Lawrence, KS (US)

(72) Inventors: Cory Berkland, Lawrence, KS (US); Mario Rivera, Lawrence, KS (US); Kate Eshelman, Lawrence, KS (US); Jian Qian, Lawrence, KS (US); Nashwa El-Gendy, Lawrence, KS (US)

(21) Appl. No.: 15/481,556

(22) Filed: Apr. 7, 2017

Related U.S. Application Data
(63) Continuation of application No. PCT/US15/54627, filed on Oct. 8, 2015.
(60) Provisional application No. 62/062,779, filed on Oct. 10, 2014.

(51) Int. Cl. A61K 31/785

(52) U.S. Cl. CPC A61K 31/785

(57) ABSTRACT

A polymeric metal sequestrant providing antibiotic activity is provided. The polymeric metal sequestrant comprises a polyamine polymer covalently coupled to a chelator, wherein the chelator has a benzene ring with more than one hydroxyl group at any position that is free. The polymeric metal sequestrant is effective in inhibiting and preventing bacterial infections and displays synergistic effects in combination with traditional antibiotics.

### CROSS-REFERENCE TO RELATED APPLICATIONS

This application is continuation of International Application No. PCT/US15/54627, filed Oct. 8, 2015, which was published as International Publication No. WO16/057754, which claims the benefit of U.S. Provisional Application No. 62/062,779 which was filed on Oct. 10, 2014 and is incorporated by reference herein in its entirety.

### BACKGROUND

Pseudomonas aeruginosa is a quintessential example of a problematic Gram-negative pathogen that can cause a wide range of human infections. It is a frequent cause of acute life-threatening infections such as burn wounds and chronic infections, for example in the lungs of cystic fibrosis (CF) patients. P. aeruginosa is also notorious for developing resistance to antimicrobial agents and continues to cause serious public health problems worldwide. Iron is an essential nutrient needed as a co-factor in bacterial respiration, nitrogen fixation, photosynthesis, and DNA synthesis and repair. Sequestration of iron from the local environment or depletion from bacterial iron storage represents a feasible antimicrobial strategy. Iron depletion has been shown to weaken bacteria and produce an adjuvant effect if combined with antibiotics.

Some small molecular iron chelators have shown an antimicrobial effect. Deferasirox, an FDA approved iron chelator for the treatment of chronic iron overload, showed a synergistic effect against Vibrio vulnificus infections combined with standard antibiotics such as ciprofloxacin. Deferasirox can cause serious damage to the kidneys or liver or severe bleeding in the stomach or intestines, and was the second drug on the list of ‘Most frequent suspected drugs in reported patient deaths’ compiled by the Institute for Safe Medical Practices in 2009. In some studies, EDTA exhibited activity against gram-positive bacteria but was much less effective against gram-negative bacteria. Conversely, EDTA was also found to increase P. aeruginosa biofilm formation when used alone at certain concentrations. Other low molecular weight iron chelators have also demonstrated evidence of toxicity at near therapeutic doses, but results tend to be highly variable and can depend on testing conditions.

For the topical treatment of bacterial infections, small molecule iron chelators might have marginal effect against common gram negative infections and the small molecular size could possibly allow absorption through skin tissue. Thus, there is a need for a safe, non-absorbable chelator with the ability to bind and sequester iron for treatment of bacterial infections, such as P. aeruginosa infections on burns and wounds.

### SUMMARY

The present disclosure is based on the discovery that certain polymeric materials are highly effective against bacterial infections and furthermore, provide a synergistic effect when combined with antibiotics. A composition is therefore provided comprising a polymer covalently coupled to one or more chelators, wherein the polymer comprises a polyamine, and wherein the one or more chelators has a benzene ring with more than one hydroxyl group at any position that is free, or a derivative of the chelator, or a salt of the chelator. The composition may further comprise an antibiotic.

In certain embodiments, the composition comprises a plurality of cross-linked polymers, wherein the polymers comprise a polyamine. In certain embodiments, the polyamine includes, but is not limited to polyallylamine (PAA), polyvinyl formamide (PVF), polyvinylamide (PVA), polylysine (PLL), polyethylenimine (PEl), or the like.

In certain embodiments, the chelator is capable of chelating a metal, a heavy metal, and more specifically, one or more of aluminum, arsenic, cadmium, chromium, copper, iron, lead, manganese, and mercury. In one particular embodiment, the chelator is 2,3 dihydroxybenzoic acid (DHBA). In another particular embodiment, the chelator is 2,3 dihydroxybenzaldehyde.

In certain embodiments, the chelator is covalently coupled to a primary amine of the polyamine through an amide bond. In certain embodiments, the chelator is covalently coupled to a primary amine of the polyamine through an amine bond. The chelator may be present in the composition in an amount to provide a molar ratio of chelator to amine of about 5% to about 40%.

In one particular embodiment, the polyamine is polyallylamine and the chelator is 2,3 dihydroxybenzoic acid. In one particular embodiment, the polyamine is polyallylamine and the chelator is 2,3 dihydroxybenzaldehyde. In one particular embodiment, the polyamine is polylysine and the chelator is 2,3 dihydroxybenzoic acid. In one particular embodiment, the polyamine is polylysine and the chelator is 2,3 dihydroxybenzaldehyde. In another particular embodiment, the polyamine is polyethylenimine and the chelator is 2,3 dihydroxybenzoic acid. In yet another particular embodiment, the polyamine is polyethylenimine and the chelator is 2,3 dihydroxybenzaldehyde.

In certain embodiments, the antibiotic is selected from the group comprising penicillin, doxycycline, ciproflaxin, kanamycin, raxibacumab, metronidazole, erythromycin, amoxicillin, ceftriaxone, gentamicin, ampicillin, tetracycline, vancomycin, streptomycin, cephalosporin, azithromycin, and rifampicin.

In certain embodiments, the composition is a hydrogel. In certain other embodiments, the composition is a topical formulation.

The present disclosure also provides a method for treating a subject with a bacterial infection. The method comprises administering a composition to a site on the subject harboring the bacterial infection, wherein the composition comprises a polymer covalently coupled to a chelator, wherein the polymer comprises a polyamine, and wherein the chelator has a benzene ring with more than one hydroxyl group at any position that is free. In another embodiment, the composition used in the method comprises a plurality of cross-linked polymers, wherein the polymers comprise a polyamine, with one or more chelators covalently coupled to one or more primary amines, respectively, of the polyamine through one or more amide bonds or amine bonds, wherein each of the one or more chelators has a benzene ring with more than one hydroxyl group at any position that is free, or a derivative of the chelator, or a salt of the chelator. In particular embodiments, the polyamine includes, but is not limited to polyallylamine (PAA), polyvinyl formamide (PVF), polyvinylamide (PVA), polylysine (PLL), polyethylenimine (PEl), or the like. In any of the above embodiments of the method, the chelator is capable of chelating a metal, a heavy metal, and more specifically, one or more of aluminum, arsenic, cadmium, chromium, copper, iron, lead, manganese, and mercury. In one particular embodiment, the chelator is 2,3 dihydroxybenzoic acid (DHBA). In another particular embodiment, the chelator is 2,3 dihydroxybenzaldehyde. In any of the above embodiments, the composition used in the method is a hydrogel.

In certain embodiments, the site is an external wound on the subject. In particular embodiments, the site is a mucosal surface such as bronchial, endometrial, gastric, penile, vaginal, olfactory, intestinal, anal, or oral.

In certain embodiments, the bacterial infection is caused by a gram-negative bacteria, such as pseudomonas aeruginosa. In certain embodiments, the bacterial infection is in the form of a biofilm.

In certain embodiments, the administering step is performed by applying the composition topically to a wound or burn on the skin or to an infection on a mucosal surface.

In any of the above embodiments, the composition used in the method may further comprise an antibiotic.

The compositions of the present disclosure may also be used to prevent bacterial infections in subjects at risk for exposure to a bacterial or having a wound that is susceptible to bacterial infection.

The present disclosure further provides a process for preparing a polymeric composition. The process comprises the following steps: obtaining a first solution comprising an activated chelator; obtaining a second solution comprising a polymer and a cross-linker, wherein the polymer comprises a polyamine; adding the first solution to the second solution at a desired ratio to form a third solution; mixing the third solution until it is transparent; and incubating the third solution at room temperature. In certain embodiments of the process, the activated chelator comprises 2,3-dihydroxybenzoic acid activated with N-hydroxysuccinimide. In certain embodiments, the polyamine is selected from the group consisting of polyallylamine, polyvinyl formamide, polyvinylamide, polylysine, and polyethylenimine. In certain embodiments, the cross-linker is N,N-methylene bisacrylamide.

In any of the above embodiments of the process, the first solution is added to the second solution in an amount sufficient to provide a molar ratio of chelator to amine of from about 5% to about 40%.

In any of the above embodiments of the process, the third solution is incubated at room temperature for at least 48 hours.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Some specific example embodiments of the disclosure may be understood by referring, in part, to the following description and the accompanying drawings.

FIG. 1 provides the reaction scheme for synthesis of cross-linked PAI-DHBA polymer.

FIG. 2 provides the iron affinity indexes of PAI-DHBA polymers as measured using a ligand competition assay.

FIG. 3 provides iron sequestration capacities of PAI-DHBA polymers, expressed as mg Fe/g PAI-DHBA. (▪) Theoretical Fe Sequestration Capacity; (▾) Experimental Fe Sequestration Capacity.

FIG. 4 provides the results of metal selective studies for essential metals, expressed as mmol metals/g PAI-DHBA.

FIG. 5 provides the results of metal selectivity studies for PAI-DHBA polymers in M63 media.

FIG. 6 provides bacterial growth curves of P. aeruginosa grown in M63 media after incubation of the media with different concentrations of PAI-DHBA for 20 min prior to starting the growth curve experiment. () 0 mg/mL; (∘) 1 mg/mL; (▾) 10 mg/mL; (Δ) 20 mg/mL.

FIG. 7 provides bacterial growth curves of P. aeruginosa grown in M63 media after incubation of the media with PAI-DHBA for 20 min or 12 h prior to starting the growth curve experiment. () (−) PAI-DHBA; (∘) 20 min; (▾) 12 h.

FIG. 8 provides the spectrophotometric analysis (UV spectra) of the polymer treated (right) and untreated (left) M63 media during bacterial growth.

FIG. 9 provides bacterial counts after bacterial incubation with different concentrations of PAI-DHBA in M63 media for 12 h.

FIG. 10 provides bacterial count after 5, 6 and 12 h of incubation of P. aeruginosa grown in M63 media with PAI-DHBA compared to the well-known iron chelating agent; EDTA. The iron chelating agents were added with bacteria to the M63 during log phase.

FIG. 11 provides bacterial counts after 5, 6, 7 and 9 h of incubation with or without PAI-DHBA and with or without addition of Ciprofloxacin (1 μg/mL) at the 5^{th} hour.

FIG. 12 provides bacterial counts after 5, 6, 7 and 9 h of incubation with or without the addition of PAI-DHBA and/or Ciprofloxacin (1 μg/mL) at the 5^{th} hour.

FIG. 13 provides bacterial counts after 5, 6, 7 and 9 h of incubation with or without PAI-DHBA and with or without addition of Gentamicin (24 μg/mL) at the 5^{th} hour.

FIG. 14 provides bacterial counts after 5, 6, 7 and 9 h of incubation with or without the addition of PAI-DHBA and/or Gentamicin (24 μg/mL) at the 5^{th} hour.

FIG. 15 provides bacterial counts after incubation with or without PAI-DHBA and with or without addition of Ciprofloxacin (1 μg/mL) at the 12^{th} hour.

FIG. 16 provides bacterial counts after incubation with or without the addition of PAI-DHBA and/or Ciprofloxacin (1 μg/mL) at the 12^{th} hour.

FIG. 17 provides the optical densities (600 nm) of M63 media inoculated with P. aeruginosa at hourly timepoints from 0-12 hours after being incubated with 20 mg of polymer for either 20 min or 12 h prior to inoculation.

FIG. 18 provides the log CFU/mL of M63 media inoculated with P. aeruginosa at timepoints from 0-12 hours after being incubated with 20 mg of polymer for either 20 min or 12 h prior to inoculation.

FIG. 19 depicts the structure of various polymeric metal sequestrants of the present disclosure. Structure I is an exemplary polymeric metal sequestrant comprising polyallylamine covalently coupled to 2,3 dihydroxybenzaldahyde. Structure II is an exemplary polymeric metal sequestrant comprising polyethylenimine covalently coupled to 2,3 dihydroxybenzaldahyde. Structure III is an exemplary polymeric metal sequestrant comprising polylysine covalently coupled to 2,3 dihydroxybenzaldahyde.

FIG. 20 provides bacterial counts of P. aeruginosa, expressed as log CFU/mL, incubated in M63 media treated with different concentrations of polymer plus conventional antibiotic (Cipro).

FIG. 21 provides bacterial counts of P. aeruginosa, expressed as log CFU/mL, incubated in M63 media treated with different concentrations of polymer plus conventional antibiotic (Genta).

FIG. 22 provides bacterial counts of P. aeruginosa, expressed as log CFU/mL, incubated in M63 media treated with different concentrations of conventional antibiotic (Cipro) plus polymer.

FIG. 23 provides bacterial counts of P. aeruginosa, expressed as log CFU/mL, incubated in M63 media treated with different concentrations of conventional antibiotic (Genta) plus polymer.

FIG. 24 provides bacterial counts, of P. aeruginosa, expressed as log CFU/mL, incubated in M63 media treated with different iron chelators or the polymer at timepoints between 5-12 h.

FIG. 25 provides the structures of polymers screened for antimicrobial activity.

FIG. 26 provides bacterial growth, expressed as Absorbance at 650 nm measured at various timepoints from 0-72 h, for cultures treated with 1-500 μg/mL of PLL-DHBA.

FIG. 27 provides bacterial growth, expressed as Absorbance at 650 nm measured at various timepoints from 0-72 h, for cultures treated with 1-500 μg/mL of PEI-DHBA.

FIG. 28 provides bacterial growth, expressed as Absorbance at 650 nm measured at various timepoints from 0-72 h, for cultures treated with 1-500 μg/mL of PAAm-DHBA.

FIG. 29 provides bacterial growth, expressed as Absorbance at 650 nm measured at various timepoints from 0-72 h, for cultures treated with 1-500 μg/mL of kanamycin.

FIG. 30 provides bacterial growth, expressed as Absorbance at 650 nm measured at various timepoints from 0-72 h, for cultures treated with 1-500 μg/mL of each of PLL-DHBA and kanamycin.

FIG. 31 provides bacterial growth, expressed as Absorbance at 650 nm measured at various timepoints from 0-72 h, for cultures treated with 1-500 μg/mL of each of PEI-DHBA and kanamycin.

FIG. 32 provides bacterial growth, expressed as Absorbance at 650 nm measured at various timepoints from 0-72 h, for cultures treated with 1-500 μg/mL of each of PAA-DHBA and kanamycin.

### DESCRIPTION

The present disclosure provides a composition that is effective in the treatment of bacterial infections. The composition is a polymeric metal sequestrant (i.e. polymers that bind and retain metals such as iron). Polymers, especially cross-linked polymeric materials, cannot be absorbed through skin, thereby limiting concerns of toxicity that have plagued the prior art. Moreover, the combination of polymeric metal sequestrant with traditional antibiotics provide a synergistic therapeutic effect and reduce the minimum inhibitory concentrations (MICs) of antibiotics.

The polymeric metal sequestrants described herein are designed with several key features such as high affinity, a large binding capacity, and selectivity for iron. Polymers mimicking the structure of a high affinity iron chelating siderophores produced by bacteria (e.g. enterobactin) were used in certain embodiments. In one embodiment, primary amine groups on polyallylamine (PAI or PAA) were simultaneously cross-linked by, for example, methylenebisacrylamide (MBA) and conjugated with 2,3-dihydroxybenzoic acid (DHBA) molecules or 2,3 dihydroxybenzaldehyde molecules, which serve as iron chelation sites. The resultant iron-sequestering polymer demonstrates strong affinity and high selectivity for iron. Further, the polymeric metal sequestrants are demonstrated herein as an effective antibiotic against P. eruginosa alone and in combination with traditional antibiotics such as ciprofloxacin, gentamycin, and kanamycin, which are commonly used for treatment of bacterial infections, especially those caused by P. aeruginosa.

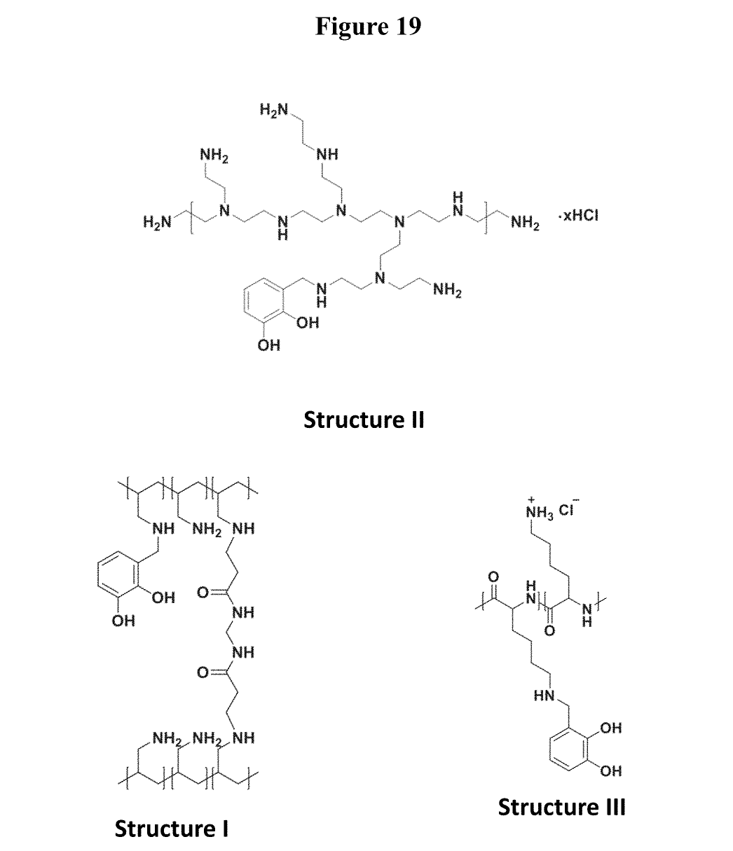

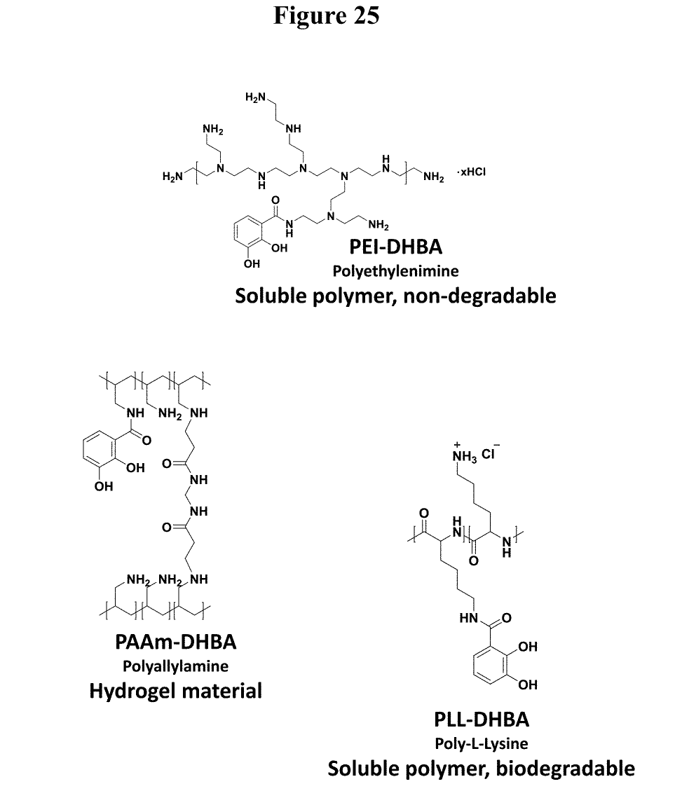

The polymeric metal sequestrants of the present disclosure comprise a polymer covalently coupled to a chelator, wherein the polymer comprises a polyamine. In some embodiments, the chelator coupled to the polymer may include 2,3 dihydroxybenzoic acid (DHBA) and other iron chelators, such as 2,3 dihydroxybenzaldehyde. FIG. 19 depicts various polymers comprising PAA, PLL, or PEI each covalently coupled to 2,3 dihydroxybenzaldehyde providing exemplary polymeric metal sequestrants of the present disclosure. FIG. 25 depicts various polymers comprising PAA, PLL, or PEI each covalently coupled to 2,3 dihydroxybenzoic acid providing additional exemplary polymer metal sequestrants of the present disclosure. DHBA acid is a fraction of the natural iron chelator Enterobactin (Log K=52) which is a high affinity siderophore that acquires iron for microbial systems. Chelators of other metals, including heavy metals that can be coupled to a polymer may also be included. As used herein, “heavy metals” are chemical elements with a specific gravity that is at least 5 times the specific gravity of water. In certain embodiments, the polymeric metal sequestrants selectively bind iron.

In some embodiments, the chelator may be coupled to the polymer via a carboxyl group of the chelator. In some embodiments, the chelator may be coupled to the polymer via a peptide bond. In some embodiments, the chelators can include a feature for coupling with the polymer, such as carboxy groups that can be coupled to the amines of the polymer through amide bonds. In other embodiments, the chelator, such as or 2,3 dihydroxybenzaldehyde, can be coupled to the amines of the polymer through an amine bond. Other crosslinking or coupling reagents can be included in the polymer and chelator system in order to prepare a polymeric chelator having the ability to chelate iron.

In some embodiments, the present disclosure provides a polymeric chelator, polymer or hydrogel, made by reacting 2,3 dihydroxybenzoic acid (DHBA) or 2,3 dihydroxybenzaldehyde to a polymer comprising a polyamine. In some embodiments, the polymeric chelators, in polymer or hydrogel form, can be fabricated as solids or equilibrated in aqueous solution as a solution or suspension. In some examples the polyamine polymer may comprise PVAm and PAAm. PVAm and PAAm are polycation hydrogels consisting of reactive primary amine side groups for the conjugation of DHBA. Cross-linked PVAm hydrogel may be synthesized by hydrolyzing a precursor polymer, PNVF, in a basic medium. Cross-linked PAAm hydrogel mat be synthesized by cross-linking the precursor PAAm chains.

In other embodiments, thioglycolic acids (TGA) in combination with the siderophore moiety dihydroxybenzoic acid (DHBA) may be introduced onto PAAm and PVA to from the polymeric chelator.

In certain embodiments, the polymeric metal sequestrants of the present disclosure comprise a plurality of cross-linked polyamine-containing polymers covalently coupled to one or more chelators that form hydrogels. The polymeric metal sequestrants, including all embodiments disclosed herein, may comprise a swelling ratio of from about 5 to about 20, or alternatively less than 5 wherein the swelling ratio is determined by (Wₛ−W_{d})/W_{d} where Wₛ and W_{d} represent the weight of polymer after full swelling in PBS, and the weight of dried polymer, respectively.

The polymeric metal sequestrants, including all embodiments disclosed herein, may further comprise a molar ratio of chelator to amine of from about 5% to about 40%, from about 10% to about 30%, from about 20% to about 25%, and any range there between. In other embodiments, the molar ratio of chelator to amine is 30%, 35% or 40%.

The polymeric metal sequestrants, including all embodiments disclosed herein, possesses an iron affinity index from about 25 to about 35 and more preferably from about 28 to about 32. Determination of the iron affinity index is described in the Examples herein below.

The polymeric metal sequestrants, including all embodiments disclosed herein, possesses an iron sequestration capacity of from about 5 mg Fe/g polymeric metal sequestrant to about 25 mg Fe/g polymeric metal sequestrant, and more preferably about 20 mg Fe/g polymeric metal sequestrant. Iron sequestration capacity, as used herein, describes the maximum iron adsorption by the polymeric metal sequestrants.

The polymeric metal sequestrants can be fabricated as solids, gels, pastes, liquids, such as being equilibrated in aqueous solution as a solution or suspension. The polymeric metal sequestrants may further be formulated for topical administration. Compositions for topical administration may include the polymeric metal sequestrants formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, and gelling agents, as necessary to preserve the composition and provide for a homogenous mixture. Transdermal administration of the present invention may also comprise the use of a “patch”. For example, the patch may supply one or more active substances at a predetermined rate and in a continuous manner over a fixed period of time.

In certain embodiments, the polymeric metal sequestrants can be incorporated into textiles, fabrics, absorbent members, gauze, wipes, bandages, or the like.

The polymeric metal sequestrants can be present in the compositions at a range of from about 1 mg/ml to about 2,000 μg/ml, from about 10 μg/ml to about 1,000 μg/ml, from about 20 μg/ml to about 500 μg/ml, from about 30 μg/ml to about 400 μg/ml, from about 40 μg/ml to about 300 μg/ml, from about 50 μg/ml to about 200 μg/ml, and from about 100 μg/ml to about 150 μg/ml, and any range there between.

In certain embodiments, the composition of the present disclosure further comprise and antibiotic in addition to the polymeric metal sequestrant. Suitable antibiotics of the composition include, but are not limited to penicillin, doxycycline, ciproflaxin, raxibacumab, metronidazole, erythromycin, amoxicillin, ceftriaxone, gentamicin, ampicillin, tetracycline, vancomycin, streptomycin, cephalosporin, azithromycin, and rifampicin.

The antibiotic may be dispersed in the polymeric metal sequestrant by methods known for administering antibiotics in other polymeric hydrogels. Examples for comparison could include topical gels containing erythromycin, intravaginal gels containing metronidazole, dental gels containing doxycycline, ocular gels such as those containing hyaluronan, as well as antibiotic-containing gels applied to other mucosal and skin surfaces. In many of these products, polymers are used to form a viscosified vehicle to facilitate placement or retention of the dose, which could also be achieved by the polymeric metal sequestrant.

The polymeric metal sequestrants may be formulated as a pharmaceutical composition comprising an effective amount of one or more polymeric metal sequestrants and optionally, one or more antibiotics dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one polymeric metal sequestrants and optionally, one or more antibiotics will be known to those of skill in the art in light of the present disclosure.

As used herein, “pharmaceutically acceptable carrier” includes any and all solvents, dispersion media, coatings, surfactants, antioxidants, preservatives, isotonic agents, absorption delaying agents, salts, preservatives, stabilizers, gels, binders, excipients, disintegration agents, lubricants, dyes, such like materials and combinations thereof, as would be known to one of ordinary skill in the art.

In accordance with the present invention, the composition is combined with the carrier in any convenient and practical manner, i.e., by solution, suspension, emulsification, admixture, encapsulation, absorption and the like. Such procedures are routine for those skilled in the art.

The polymeric metal sequestrants of the present disclosure can be used as an antibiotic therapy to treat or prevent bacterial infections in connection with external wounds and burns and also in the treatment or prevention of bacterial infections present on mucosal membranes. In one embodiment, the polymeric metal sequestrants are applied topically to the wound or burn surface or directly on the mucosal surface.

The polymeric metal sequestrants of the present disclosure can be used to disrupt biofilms.

In certain embodiments, a traditional antibiotic can be added to the site of bacterial infection simultaneously with the polymeric metal sequestrant or following an initial treatment with the polymeric metal sequestrant. More generally, such an agent would be provided in a combined amount with a polymeric metal sequestrant effective to kill or inhibit proliferation of the bacteria. This process may involve contacting the cell(s) with an antibiotic and the polymeric metal sequestrant at the same time or within a period of time wherein separate administration of the polymeric metal sequestrant and antibiotic to a cell, tissue or organism produces a desired therapeutic benefit. This may be achieved by contacting the cell, tissue or organism with a single composition or pharmacological formulation that includes both a polymeric metal sequestrant and one or more antibiotics, or by contacting the wound or infection site with two or more distinct compositions or formulations, wherein one composition includes a polymeric metal sequestrant and the other includes one or more antibiotics.

The polymeric metal sequestrants may be applied daily, or one to four times daily, based on the location of the infection, the severity of the infection, or the bacteria causing the infection.

It is contemplated that any embodiment discussed in this specification can be implemented with respect to any method or composition of the invention, and vice versa. Furthermore, compositions of the invention can be used to achieve methods of the invention.

### EXAMPLES

The following materials and methods were used for Examples 1-5.

### Materials

Polyallylamine (PAI; 56 kDa), 2,3-dihydroxybenzoic acid (DHBA), triethylamine (TEA), N,N′-methylenebisacrylamide (MBA), N-(3 dimethylaminopropyl)-N-ethylcarbodiimide hydrochloride (EDC), N-hydroxysuccinimide (NHS), and N,N-dimethylformamide (DMF) were purchased from Sigma-Aldrich. Ciprofloxacin hydrochloride (Cipro) was purchased from MP Biomedicals, Inc. Gentamicin sulfate (Genta), sodium ethylene diamine tetraacetate (EDTA), agar, and potassium chloride (KCl) were purchased from Fisher Scientific. Luria broth media (LB; pH 7.1) was purchased from Teknova. N,N-bis (2-hydroxybenzyl) ethylenediamine-N,N-diacetic acid (HBED) was purchased from Strem Chemicals, Inc. Sodium chloride (NaCl) was purchased from Acros Organics. Potassium dihydrogen phosphate (KH₂PO₄) and disodium hydrogen phosphate (Na₂HPO₄) were purchased from Santa Cruz Biotechnology, Inc.

### Bacterial Strains and Growth Conditions

Pseudomonas aeruginosa strain PA01 wild type (WT) purchased from the University of Washington Genome Sciences was used in all studies. P. aeruginosa was grown in M63 minimal media [2 g KH₂PO₄, 13.6 g (NH₄)₂SO₄, 2 g glucose, 5 g casamino acids, 0.25 g tryptophan, 4 g citric acid, 3 μM FeSO₄.7H₂O, and 1 mM MgSO₄ per 1 L water [pH 7.0]] modified after O'Toole and Kolter, Initiation of biofilm formation in Pseuomonas fluorescens WCS365 proceeds via multiple, convergent signaling pathways: a genetic analysis, Mol. Microbiology, 1998; 28(3):449-61. All glassware was acid-washed by soaking in nitric acid and rinsed 5 times with deionized water.

### Preparation and Characterization of Iron Sequestering Polymer

NETS-activated DHBA was synthesized before preparing the polymer. A solution of DHBA (770 mg, 5 mmol) and NETS (690 mg, 6 mmol) in 5 mL of DMF was mixed with a solution of EDC (1200 mg, 6.2 mmol) in 5 mL of DMF. The mixture was stirred at room temperature for 8 h and used for the next step without any purification. The PAI cross-linking and DHBA conjugation were conducted in a single step. Briefly, a 15% w/w PAI hydrochloride (56 kDa) solution containing a predetermined amount of N,N-methylene bisacrylamide (BMA; 5%, molar ratio of cross-linker to total amines) was prepared in H₂O/DMF (50/50 v/v) mixture. Then, the NHS-activated DHBA solution with a desired DHBA/amine molar ratio (5-40%) was added to the solution. After sonication for 2 min to get a transparent solution, TEA was added to the solution and mixed thoroughly, and then the solution was incubated at room temperature for 48 h. The cross-linked polymer gels were washed with 0.1 M sodium hydroxide for several days under the protection of nitrogen, and then lyophilized. The polymer gels were ground to powder for subsequent studies. The particle size of the ground powder was ˜100 μm measured by optical microscopy. As the polymers were cross-linked particles, the DHBA conjugation ratios could not be characterized directly by NMR analysis. Instead, the unconjugated DHBA left in the solution after the reaction was determined by NMR. The real conjugated DHBA ratio was calculated by deducting the unconjugated DHBA ratio from the feed DHBA ratio.

### Swelling Studies

The swelling behavior of cross-linked polymers was studied in PBS buffer (pH 7.4). Dried polymer samples were placed in PBS buffer at room temperature. The weight of the swollen polymer samples was determined at different time points until there was no weight gain, indicative of complete swelling. The swelling ratio was defined as the fractional increase in the weight of the cross-linked polymer due to water absorption (Park, et al., Effect of swelling ratio of injectable hydrogel composites on chondrogenic differentiation of encapsulated rabbit marrow mesenchymal stem cells in vitro, Biomacromolecules, 2009; 10(3):541-6). The swelling ratio of the cross-linked polymers was determined by the following equation:

$$Swellingratio = \frac{{W}_{s} - {W}_{d}}{{W}_{d}}$$

Here, Wₛ and W_{d} represent the weight of polymer after full swelling in PBS, and the weight of dried polymer, respectively.

### Determination of the Iron Affinity Index

The iron affinity index of the polymer was measured using a ligand competition assay. The competitive chelation of iron by the polymers in equilibrium with EDTA (a water-soluble chelator) was used to determine the affinity index. Briefly, 1.5 mL of 10 mM EDTA solution, 2 mL of 5 mM FeCl₃ solution, 21.5 mL PBS and a known mass of polymer were mixed together and rotated at 25° C. for 5 days. Then, the concentration of the soluble iron complex was determined by inductively coupled plasma optical emission spectrometry (ICP-OES; Optima 2000 DV, PerkinElmer, USA). The affinity index of the polymer was determined following the procedure reported in literature (Feng et al., Iron (III) chelating resins. VI. Stability constants of iron(III)-ligand complexes on insoluble polymeric matrices, J. Appl. Poly. Sci., 1995; 56(10):1231-7). In the equilibrium situation, the system could be represented in the following way (for brevity, all charges have been omitted):

$$Fe ( EDTA ) + 3 DHBA ⇌ {Fe ( DHBA )}_{3} + EDTA \\ Kq = \frac{[ Fe ( DHBA ) 3 ] [ EDTA ]}{{[ Fe ( EDTA ) ] [ DHBA ]}^{3}} = \frac{[ Fe ( DHBA ) 3 ]}{{[ Fe ] [ DHBA ]}^{3}} \times \frac{[ Fe ] [ EDTA ]}{[ Fe ( EDTA ) ]} \qquad ( 1 )$$

The iron stability constant of DHBA (Q) could be defined as follows:

$$Q = \frac{[ Fe ( DHBA ) 3 ]}{{[ Fe ] [ DHBA ]}^{3}} \qquad ( 2 )$$

The iron stability constant of EDTA (K) could be defined as:

$$K = \frac{[ Fe ( EDTA ) ]}{[ Fe ] [ EDTA ]} \qquad ( 3 )$$

Herein, Iron affinity index was defined as Log Q. Based on equation (1), (2) and (3):

Iron affinity index=Log Q=Log(Kq×K)=Log Kq+Log K

The value of K was known, and Kq could be easily calculated based on the iron concentration, EDTA concentration, and the concentration of DHBA groups in polymers as described in literature (Feng, et al.). The iron affinity index showed how strong the polymers chelated with iron versus EDTA.

### Determination of the Iron Sequestration Capacity

A known mass of polymer was incubated in a 5 mM FeCl₃ solution in the presence of 5 mM EDTA as a stabilizer at 25° C. for a week. The remaining iron concentration was determined by ICP-OES.

### Selectivity Study

The iron selectivity of PAI-DHBA polymer was determined in the presence of copper, zinc, manganese, calcium, nickel and potassium. A solution containing all these metal ions, each at a concentration of 0.4 mM, was prepared in a phosphate buffer at pH 7.2 containing 2 mM EDTA. A predetermined amount of polymer was added into the solution and incubated at 25° C. for 5 days. The concentration of each metal ion remaining in solution was determined by ICP-OES. For the selectivity in M63 media, a predetermined amount of polymer was added into the M63 solution and incubated at 25° C. for 3 days. The concentrations of the metals (Fe³⁺ and Mg²⁺) remaining in the solution were determined by ICP-OES.

### Effect of Iron Sequestration on Bacterial Growth

Fifty mL of M63 media containing 1 mg, 10 mg and 20 mg/mL of insoluble PAI-DHBA was incubated for 20 min with shaking (230 rpm, 37° C.). In addition, 20 mg/mL polymer was incubated for 12 h to study the effect of incubation time. After incubation, the polymer was separated by centrifugation (4,000 rpm, 4° C. and 15 min). The supernatant was transferred to acid-washed 250 mL glass Erlenmeyer flasks. A single colony of P. aeruginosa was inoculated in 5 mL LB (25 g/L, pH 7.1) and grown overnight with shaking at 230 rpm and 37° C. The overnight inoculum was centrifuged for 12 min at 4,000 rpm and 4° C. and then resuspended in 5 mL of fresh M63 media. Resuspended cells were transferred to 50 mL of polymer-treated or untreated M63 media, which resulted in an OD₆₀₀=0.01. The cells were cultured at 37° C. and 230 rpm. Samples (1-2 mL) were removed from cultures every hour to measure the OD₆₀₀. At 2, 4, 6, 8, and 10 h of growth, 100 μL of culture was serially diluted in phosphate buffer saline (PBS at pH 7.4) and plated on LB agar plates using a previously described drop plate method (Herigstad, et al., How to optimize the drop plate method for enumerating bacteria, J. Microbiological Methods, 2001; 44(2):121-9). The LB/agar plates were incubated between 16-18 h at 37° C. Single colonies were enumerated and colony-forming units per milliliter (CFU/mL) were determined.

### Influence of the Novel Iron-Sequestering Polymer on Bacterial Growth Using 24-Well Plates

One mL of M63 was used to culture the bacteria in 24 well clear flat bottom plates (Midsci). An overnight growth in LB was diluted to an OD₆₀₀=0.3. The diluted culture was then added to the M63 media for a starting OD₆₀₀=0.003. The 24-well plate was wrapped with Parafilm to avoid evaporation. After incubating at 37° C. and 40 rpm for 12 h, entire wells were serially diluted in PBS (pH 7.4) and plated on LB agar by the drop plate method. The plates were incubated between 16-18 h at 37° C. CFU/mL was determined by counting single colonies. To compare different concentrations of the polymer, the reported amount of PAI-DHBA was added with the bacteria at the beginning of the experiment.

### Comparison to Traditional Iron Chelating Agents

Test chelators including 20 mg PAI-DHBA and 212 mg HBED (equivalent to 500 μM) were added to the media before bacterial inoculation. The CFU/mL was determined after 5, 6 and 12 h of incubation.

### PAI-DHBA as an Adjuvant to Conventional Antibiotics on Bacterial Growth

Ciprofloxacin or Gentamicin was added to the culture after 5 h of incubation. The polymer (20 mg/mL) was added to the media directly before culturing cells or with the antibiotics after 5 h of growth. At different time intervals of incubation (5, 6, 7 and 9 h), entire wells were serially diluted and plated on LB agar to determine CFU/mL.

Ciprofloxacin was also added to 1 mL cultures after 12 h of incubation. The polymer (20 mg/mL) was added to media with the addition of bacteria or simultaneously with Ciprofloxacin. At the time intervals of 12, 13, 14, 16 and 24 h, entire wells were serially diluted and plated on LB agar to determine CFU/mL.

### Example 1: Synthesis and Characterization of PAI-DHBA Polymer

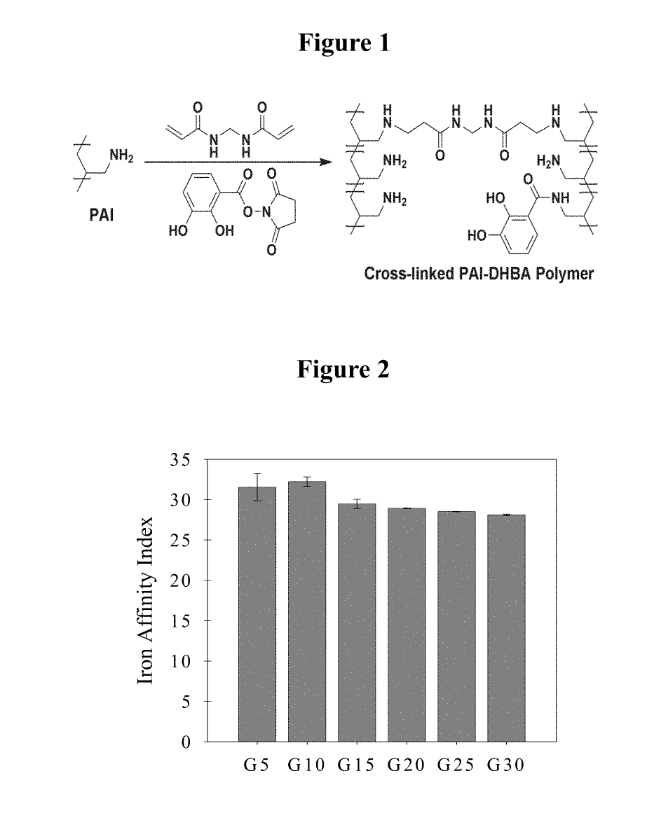

Previous preparations of PAI-DHBA polymer used a two-step synthesis strategy. PAI hydrochloride was first cross-linked with N,N-methylene bisacrylamide (BMA) by a Michael-type addition reaction and then the formed PAI hydrogel was further conjugated to DHBA via EDC/NHS conjugation chemistry. This two-step strategy was time consuming, and in the second step, DHBA conjugation may be favored near the particle surface. In this report, the polymer cross-linking and DHBA conjugation were conducted in a single step. DHBA conjugation was controlled by adjusting the DHBA/polymer feed ratios. Several PAI-DHBA polymers with various DHBA content (5-40% of total amines) but the same cross-linking density (5%) were prepared via this one step strategy as shown in FIG. 1. DHBA conjugation ratios which are shown in Table 1 were determined by NMR analysis. As the DHBA content increased from 5% to 30%, the swelling ratios decreased from 11.8 to 5.3, indicating that the gel became more hydrophobic as DHBA conjugation increased. When incubated with Fe³⁺ solution, all the PAI-DHBA samples exhibited dark color indicating chelation with Fe³⁺, while the PAI gel did not show a color change.

TABLE 1
Synthesis and characterization of PAI-DHBA polymers.

| | Cross-linking | Feed | Found | Swelling ratio |
|---|---|---|---|---|
| Sample | Density^{a} | DHBA/amine^{b} | DHBA/amine^{c} | pH 7.4 |
| G0 | 0.05 | 0 | 0 | 17.2 ± 2.1 |
| G5 | 0.05 | 0.05 | 0.0311 | 11.8 ± 1.9 |
| G10 | 0.05 | 0.10 | 0.0700 | 8.2 ± 1.1 |
| G15 | 0.05 | 0.15 | 0.1117 | 6.9 ± 0.8 |
| G20 | 0.05 | 0.20 | 0.1488 | 5.6 ± 1.2 |
| G25 | 0.05 | 0.25 | 0.1743 | 5.4 ± 0.6 |
| G30 | 0.05 | 0.30 | 0.2216 | 5.3 ± 0.4 |
| G35 | 0.05 | 0.35 | 0.2689 | — |
| G40 | 0.05 | 0.40 | 0.3216 | — |

^{a}Feed molar ratio of cross-linker to total amines
^{b}Feed molar ratio of DHBA to total amines
^{c}Found molar ratio of DHBA to total amines by a modified NMR analysis

The strength of iron chelation is an important parameter for iron chelating materials; however, affinity cannot be calculated for materials in the conventional sense. Since the polymers are cross-linked particles, the chelation between the polymer materials and iron ions presents a heterogeneous system and direct equilibrium constants are not obtainable. Thus, the term iron affinity index was used to assess how strong the polymers bind and trap iron relative to a reference iron chelator with a documented stability constant. The iron affinity index was determined by a ligand competition method in equilibrium with EDTA (iron stability constant 10²⁵). The iron affinity index was calculated based on the equation for the calculation of stability constant as described in the preceding materials and methods part entitled “Determination of the Iron Affinity Index”. All the polymers with various DHBA contents showed higher iron affinity indices than EDTA (FIG. 2). The G10 sample had the highest iron affinity index (32.2), which indicated that the iron affinity of G10 polymer is 10⁷ times stronger than EDTA (Log stability constant is 25.1, also shown in FIG. 2 for comparison). For all the other samples tested, they all showed at least 10³ times stronger of iron affinity than EDTA. As the DHBA content increased from 5 to 30%, the affinity indexes of polymers decreased from 32.2 to 28.1. It should be noted that, theoretically, all the samples should have almost the same affinity indices, since the intrinsic affinity indexes of the DHBA groups in different samples were the same. As the DHBA content increased; however, the increased hydrophobicity of the polymers may have hindered Fe³⁺ access or coordination, hence reducing the apparent iron affinity indexes based on the calculation.

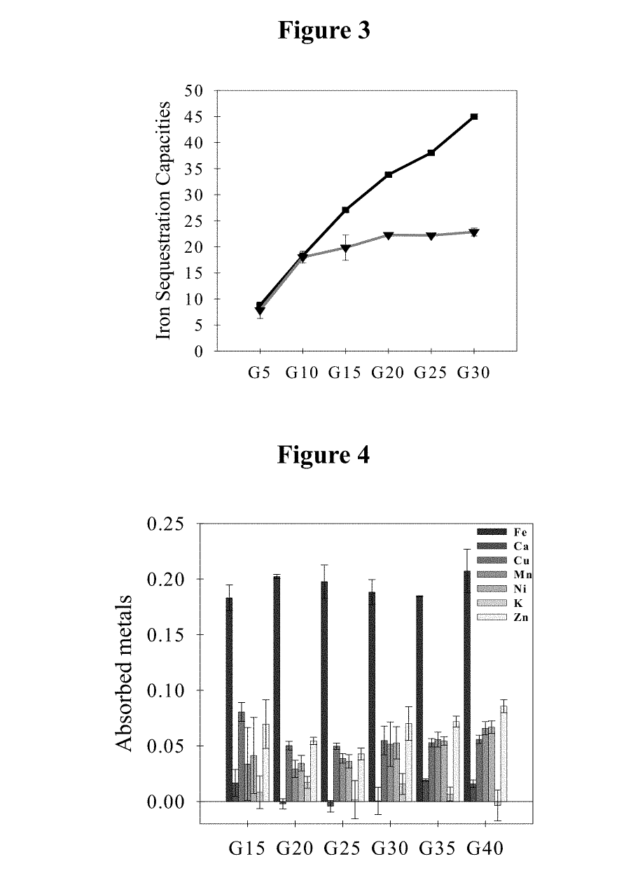

The iron sequestration capacity, which describes the maximum iron adsorption by the polymers, was also investigated. In order to reach the maximum iron sequestration, all the samples were incubated in a Fe³⁺ solution for one week. The theoretical and experimental iron sequestration capacities of the polymers with various DHBA contents were determined (FIG. 3). As the DHBA content increased, the experimental iron sequestration capacities also went up for low DHBA conjugation (5-20%), and reached a plateau (20-30%) around 20 mg Fe/g polymer. For all the samples tested, only the samples with low DHBA content achieved the theoretical iron sequestration capacities. The increased hydrophobicity of the polymers at higher DHBA conjugation percentages probably limited Fe³⁺ access to the gel particle interior. It is noteworthy that after the polymers containing chelated iron were incubated with fresh PBS containing 2 mM EDTA for 1 week, iron was not detectable in the medium by ICP-OES (data not shows indicating that iron sequestration by the polymers is not reversible.

### Example 2: PAI-DHBA Exhibited High Selectivity for Iron

Selectivity to iron is especially important for the application of iron sequestering polymers in the biological field. Poor selectivity may affect the bioavailability or the balance of essential metal ions such as Cu²⁺, Zn²⁺, Ca²⁺, Mn²⁺, Ni²⁺, or K⁺. The influence of other metals on the sequestration of Fe³⁺ by the polymers was investigated using a multi-metal system. The concentration of each metal was fixed at 0.4 mM, and the metal/polymer ratio was fixed at 0.2 mmol per gram of polymer. All the samples absorbed almost 100% of the iron present in the media while typically the absorption for other essential metals was considerably lower, demonstrating high selectivity for iron (FIG. 4).

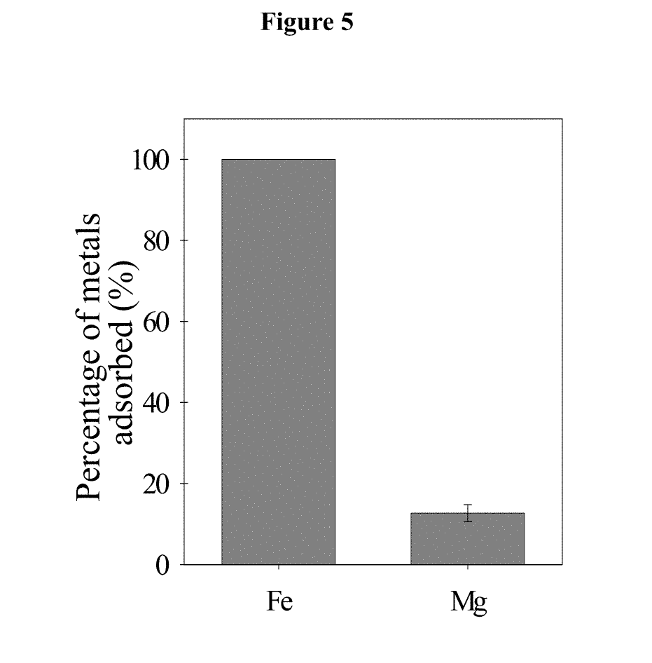

The selectivity of the polymer (G25) was also tested in the M63 media used in the P. aeruginosa studies. The M63 media only contained Fe³⁺ and Mg²⁺ metals. All the Fe³⁺ and only about 12% of the Mg²⁺ in the solution were sequestered by the polymer (FIG. 5). When considering swelling of the polymer, Mg²⁺ is likely primarily physically absorbed with imbibed water, rather than specifically chelated.

### Example 3: Iron Depletion from M63 Using PAI-DHBA Inhibits Bacterial Growth

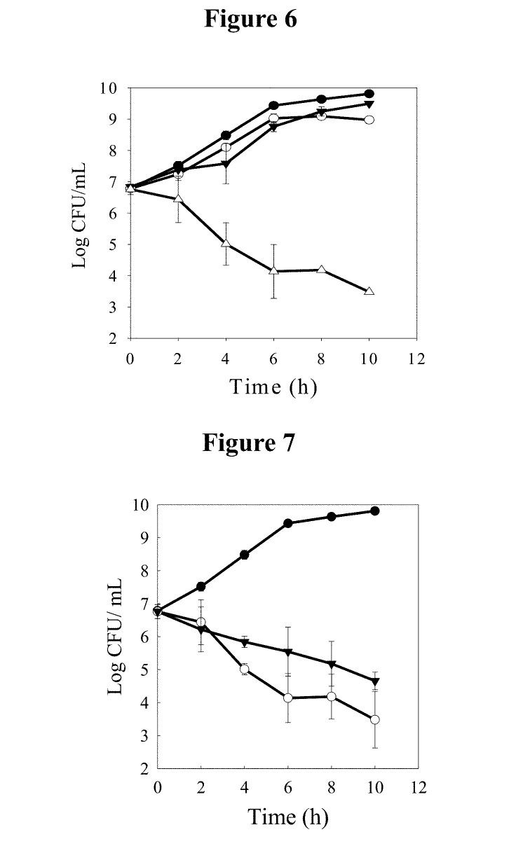

The polymer G25 was selected for studies with P. aeruginosa. Iron depletion from the medium was achieved by incubating PAI-DHBA in M63 for 20 min. The media was then used to test bacterial growth compared to growth in untreated media. Three concentrations of the novel polymer were used in this experiment (1, 10 and 20 mg/mL) to determine the appropriate concentration for subsequent studies. Bacterial growth, in normal and iron-depleted environments, was monitored by enumeration of colony forming units (CFUs) (FIG. 6). There were small differences in cultures treated with 1 mg or 10 mg per mL media. In contrast PAI-DHBA at 20 mg/mL showed not only growth inhibition, but bacterial cell death as well. It is important to note that the concentration of iron in the prepared media was 4.3 μM and after 20 min incubation with 20, 10, and 1 mg of PAI-DHBA, the iron concentration left in media was analyzed and found to be undetectable, 0.5 μM, and 1.2 μM, respectively.

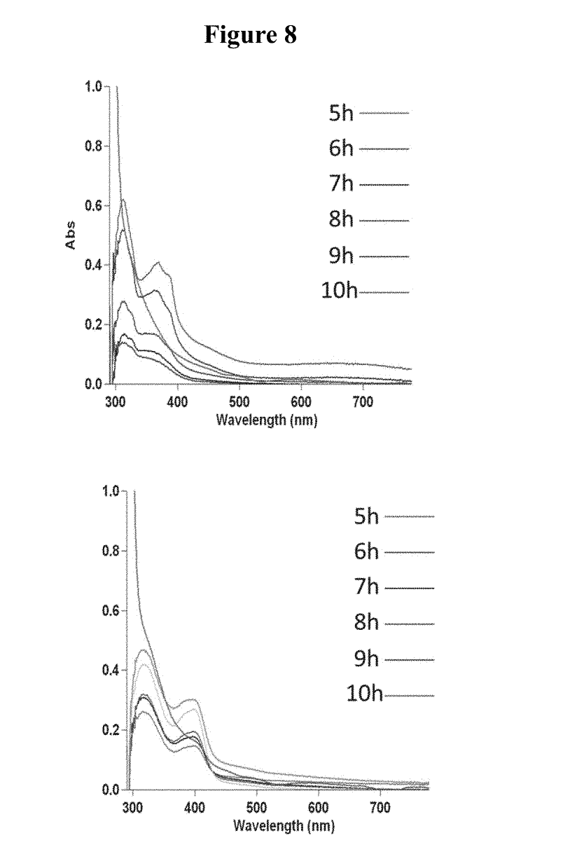

At the 9^{th} h of incubation, a greenish color was observed in the untreated media while the polymer-treated media showed a yellow color at the 6^{th} h of incubation. The intensity of these colors gradually increased with incubation time as confirmed by spectrophotometric measurements of the absorbance of the samples (FIG. 8). The greenish color indicated the release of pyocyanin (PYO), which absorbs at 690 nm and 368 nm. The yellow color indicated the release of pyoverdin (the major iron siderophore produced by P. aeruginosa), which absorbs at 400 nm. PYO is one of the predominant phenazines secreted by P. aeruginosa in the early stationary phase and has been shown to act as both an important virulence factor and a signaling molecule. The release of pyoverdin in the polymer-treated M63 is consistent with iron starvation, since pyoverdin is the major siderophore secreted by P. aeruginosa to procure iron under depletion conditions.

The time required for the polymer to sequester iron from the media was studied by incubating 20 mg polymer with M63 for 20 min and 12 h. Incubating the polymer with the growth media for 20 min prior to cell culture had the same effect as 12 h, indicating rapid iron chelation and sequestration. In both cases, bacterial growth was inhibited and cell death was observed (FIG. 7). Iron sequestering polymers offer a potent approach to prevent bacterial colonization and growth.

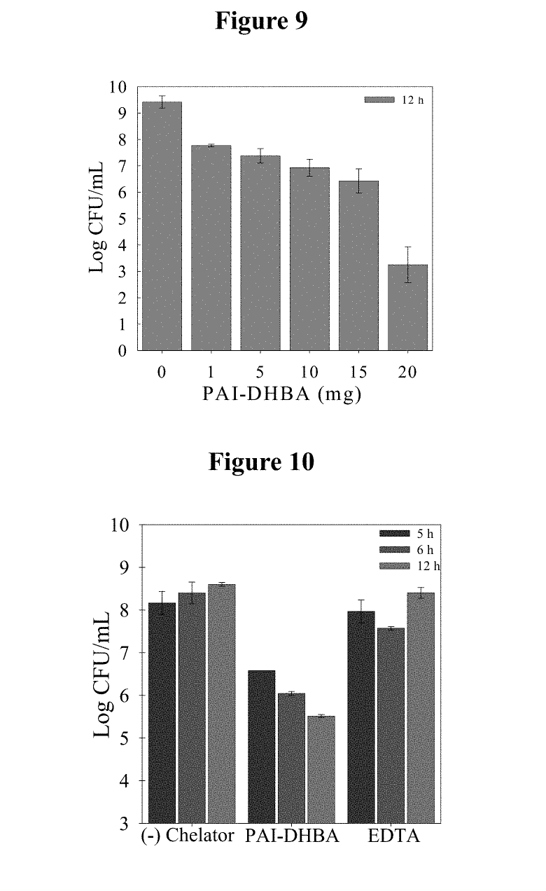

To investigate the minimum concentration of polymer needed to inhibit P. aeruginosa growth when delivered during inoculation, PAI-DHBA (0, 1, 5, 10, 15, 20 mg) was added to 1 mL M63 in 24 well plates, and dispersed to form a homogeneous suspension. After 12 h, bacterial growth significantly decreased as the concentration of the iron-chelating polymer was increased from 1 mg/mL to 20 mg/mL (FIG. 9). The polymer at a concentration of 20 mg/mL reduced the viable bacteria by approximately 10⁶ CFU/well. Thus, introducing PAI-DHBA during inoculation prevented the growth of P. aeruginosa. The wells were also visually examined 12 h after treatment. The untreated culture (control) showed a greenish color compared to the blank indicating the release of phenazines. On the contrary, cultures treated with PAI-DHBA did not show an obvious color change, which may be due to the retardation of bacterial growth.

### Example 4: PAI-DHBA is a More Potent Inhibitor of Bacterial Growth than Traditional Iron Chelators

Currently, the most commonly used drug for systemic iron chelation therapy is deferoxamine (DFO). Despite the efficacy of DFO as a chelator, the utility of this drug is limited due to high toxicity and a very short plasma half-life (˜5.5 min). Other low molecular weight iron chelators have also been explored, but they have also demonstrated evidence of toxicity at near therapeutic doses. High molecular weight or cross-linked iron chelating polymers have emerged as a promising mode of topical or non-absorbed iron chelation therapy.

In this study, the effect of a well-known low molecular weight iron chelator was studied on the growth of P. aeruginosa and compared to the effect of PAI-DHBA. EDTA, 208 mg (equivalent to 500 μM) was added to the M63 media during inoculation and the CFU/mL determined after 5, 6 and 12 h of incubation (FIG. 10). A small, transient difference between the control (no chelator), and EDTA treatments was observed after 6 h of incubation but it disappeared after 12 h, with values resembling those of untreated media. At pH 7.0, the EDTA affinity constant for Fe (III) is 10²⁵. In comparison, pyoverdin binds Fe(III) with an affinity constant of 10²⁵ thus the reversible chelation of iron by EDTA may have facilitated iron uptake via siderophores secreted by P. aeruginosa. In contrast, nearly irreversible sequestration of iron by PAI-DHBA efficiently depletes the medium from iron and inhibits bacterial growth.

### Example 5: Adjuvant Effect of Iron Sequestering Polymer on the Antimicrobial Activity of Ciprofloxacin and Gentamicin Against P. aeruginosa

Iron depletion has been shown to enhance the bactericidal effect of antibiotics, such that the combination of an iron chelator and antibiotics may have synergistic therapeutic effects. Aminoglycosides such as Gentamicin and fluoroquinolones such as Ciprofloxacin are commonly used to treat P. aeruginosa infections in clinical practice. In this study, the potential additive or synergistic effect of these antibiotics and PAI-DHBA was investigated on cultures where (i) media was treated with PAI-DHB (20 mg/mL) at the time of inoculation and supplemented with either Ciprofloxacin (1 μg/mL) or Gentamicin (24 μg/mL) after 5 h, and (ii) cultures in log phase (5 h after inoculation) were treated simultaneously with polymer and antibiotic at the indicated concentrations. The concentrations of antibiotics reflect reported MICs against P. aeruginosa.

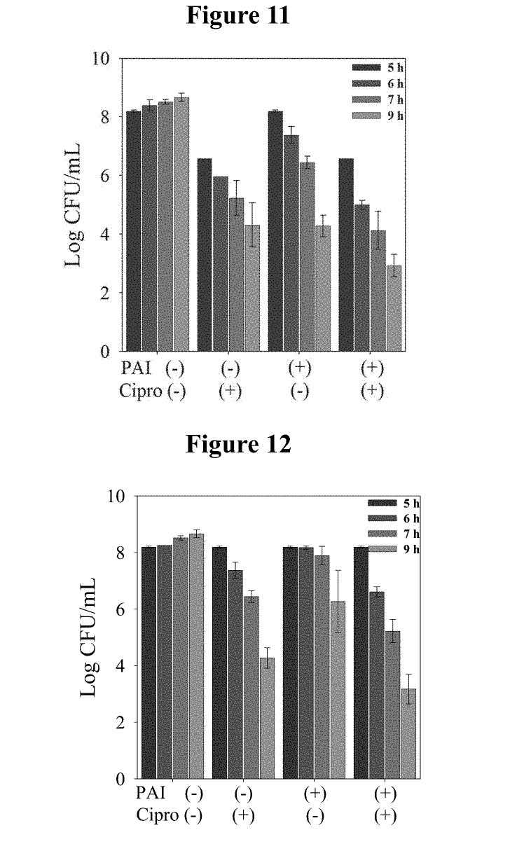

Results obtained from treating media at the time of inoculation are shown in FIG. 11: Compared to control, (PAI-DHBA(−) and Cipro(−), cultures treated with antibiotic alone, Cipro(+) show significant growth inhibition, with an overall reduction in CFU/mL of approximately 4 log units at 9 h. Treatment with polymer alone, PAI-DHBA(+), causes similar growth inhibition at the 9 h as treatment with Ciprofloxacin, except that the growth inhibition is more noticeable in the earlier hours when the polymer is present. Treatment with polymer and Ciprofloxacin causes even larger growth inhibition than treatment with antibiotic alone, or polymer alone, strongly suggesting synergistic action.

Results from treating cultures with polymer and antibiotic during log phase are shown in FIG. 12: Control and Ciprofloxacin treated cultures are very similar to those in FIG. 11. Note, however, that polymer exerts a significantly lower growth retardation effect when added to growing cultures, and although there is a synergistic effect of adding polymer and Ciprfloxacin to growing cultures, the inhibitory effect is less pronounced than treating media with polymer at the time of inoculations.

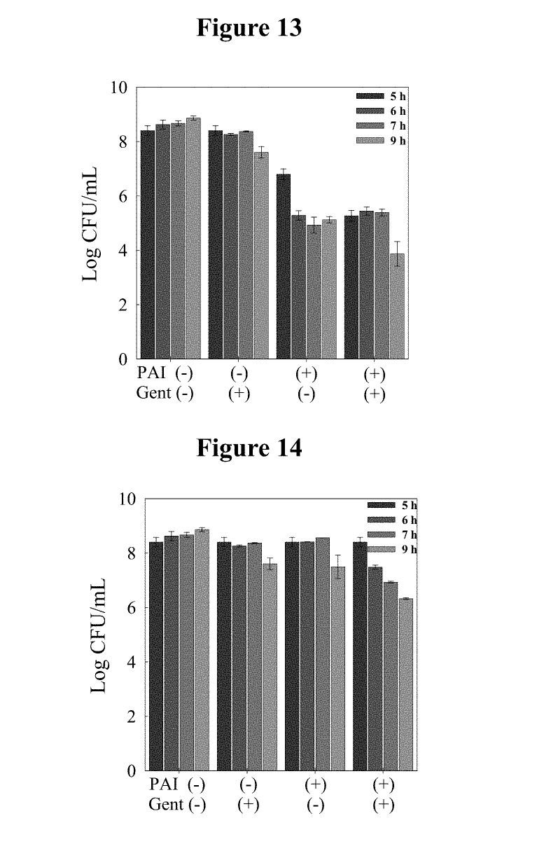

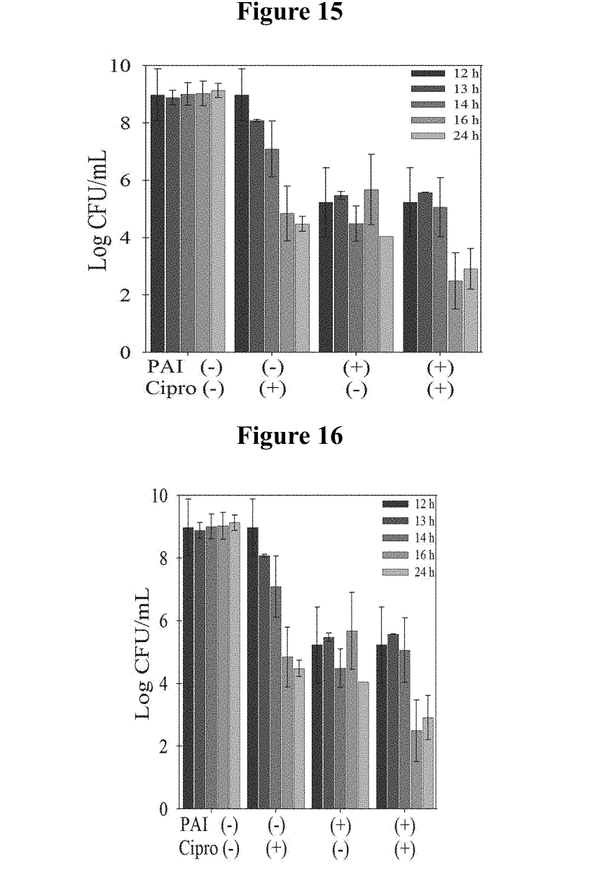

Similar experiments were carried out to study possible synergistic effects with Gentamicin (FIGS. 13, 14): When polymer is added at the time of inoculation there is significant growth retardation (FIG. 13), whereas when polymer and antibiotic are added simultaneously to growing cultures the effect is much less pronounced (FIG. 14). It is noteworthy that iron sequestration from the media by polymer is less efficient in inhibiting bacterial growth when carried out in mid log phase. Similar observations were made with Ciprofloxacin and polymer by treating cell cultures in stationary phase (12 h) (FIGS. 15, 16). The observations suggest that bacteria in growing cultures or in stationary phase may have accumulated iron in storage proteins, such as bacterioferritn, which enables growth and survival even if the medium is depleted of iron by treatment with polymer. In contrast, when the medium is iron-depleted before active cell division, bacterial cells cannot store iron and become more susceptible to iron starvation.

### Example 6: Effect of Iron Sequestration on Bacterial Growth after Different Time of Incubation

Fifty mL of M63 media containing 20 mg/mL of insoluble PAI-DHBA was incubated for 20 min and 12 h with shaking (230 rpm, 37° C.) to study the effect of incubation time. After incubation, the polymer was separated by centrifugation (4,000 rpm, 4° C. and 15 min). The supernatant was transferred to acid-washed 250 mL glass Erlenmeyer flasks. A single colony of P. aeruginosa was inoculated in 5 mL LB (25 g/L, pH 7.1) and grown overnight with shaking at 230 rpm and 37° C. The overnight inoculum was centrifuged for 12 min at 4,000 rpm and 4° C. and then resuspended in 5 mL of fresh M63 media. Resuspended cells were transferred to 50 mL of polymer-treated or untreated M63 media, which resulted in an OD₆₀₀=0.01. The cells were cultured at 37° C. and 230 rpm. Samples (1-2 mL) were removed from cultures every hour to measure the OD₆₀₀. At 2, 4, 6, 8, and 10 h of growth, 100 μL of culture was serially diluted in phosphate buffer saline (PBS at pH 7.4) and plated on LB agar plates using a previously described drop plate method (Herigstad, et al.). The LB/agar plates were incubated between 16-18 h at 37° C. Single colonies were enumerated and colony-forming units per milliliter (CFU/mL) were determined.

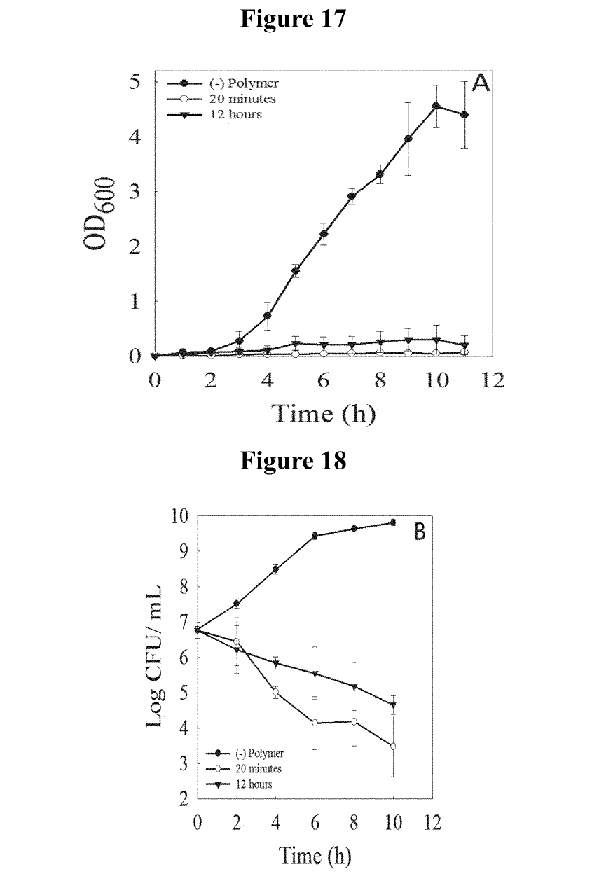

The time required for the polymer to sequester iron from the media was studied by incubating 20 mg polymer with M63 for 20 min and 12 h. Incubating the polymer with the growth media for 20 min prior to cell culture had the same effect as 12 h, indicating rapid iron chelation and sequestration. In both cases, bacterial growth was inhibited and cell death was observed (FIGS. 17, 18). Iron sequestering polymers offer a potent approach to prevent bacterial colonization and growth.

### Example 7: Different Concentrations of PAI-DHBA as an Adjuvant to Conventional Antibiotics on Bacterial Growth

One mL of M63 was used to culture the bacteria in 24 well clear flat bottom plates (Midsci). An overnight growth in LB was diluted to an OD₆₀₀=0.3. The diluted culture was then added to the M63 media for a starting OD₆₀₀=0.003. PAI-DHBA at different concentrations was added directly to the media before bacterial inoculation. Gentamycin (24 μg/mL) or Ciprofloxacin (1 μg/mL) was also added to 1 mL cultures after 5 h of incubation. The 24-well plate was wrapped with Parafilm to avoid evaporation. After incubating at 37° C. and 40 rpm for 12 h, entire wells were serially diluted in PBS (pH 7.4) and plated on LB agar by the drop plate method. The plates were incubated between 16-18 h at 37° C. CFU/mL was determined by counting single colonies. At the time intervals of 12, 13, 14, 16 and 24 h, entire wells were serially diluted and plated on LB agar to determine CFU/mL.

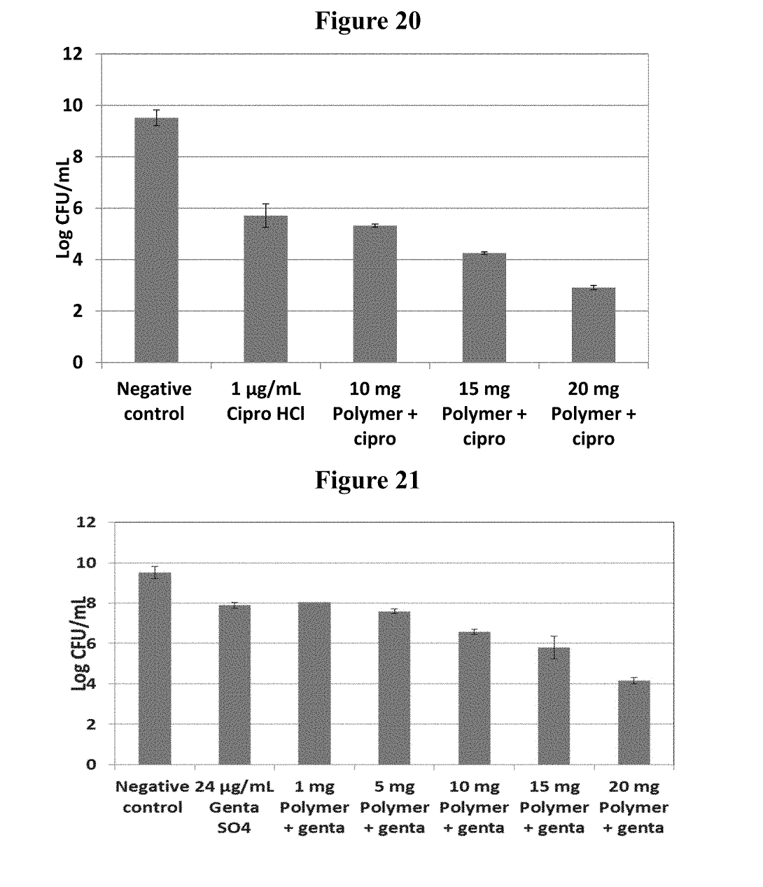

To investigate the optimal concentration of polymer needed as antibiotic adjuvant to inhibit P. aeruginosa growth, PAI-DHBA (0, 1, 5, 10, 15, 20 mg) was tested. Bacterial growth significantly decreased as the concentration of the polymer was increased from 1 mg/mL to 20 mg/mL (FIGS. 20, 21).

### Example 8: PAI-DHBA as an Adjuvant to Different Concentrations of Conventional Antibiotics on Bacterial Growth

One mL of M63 was used to culture the bacteria in 24 well clear flat bottom plates (Midsci). An overnight growth in LB was diluted to an OD₆₀₀=0.3. The diluted culture was then added to the M63 media for a starting OD₆₀₀=0.003. PAI-DHBA (20 mg/mL) was added directly to the media before bacterial inoculation. Gentamycin (6, 12 and 24 μg/mL) or Ciprofloxacin (0.1, 0.5 and 1 μg/mL) at different concentrations was also added to 1 mL cultures after 12 h of incubation. The 24-well plate was wrapped with Parafilm to avoid evaporation. After incubating at 37° C. and 40 rpm for 12 h, entire wells were serially diluted in PBS (pH 7.4) and plated on LB agar by the drop plate method. The plates were incubated between 16-18 h at 37° C. CFU/mL was determined by counting single colonies. At the time intervals of 12, 13, 14, 16 and 24 h, entire wells were serially diluted and plated on LB agar to determine CFU/mL.

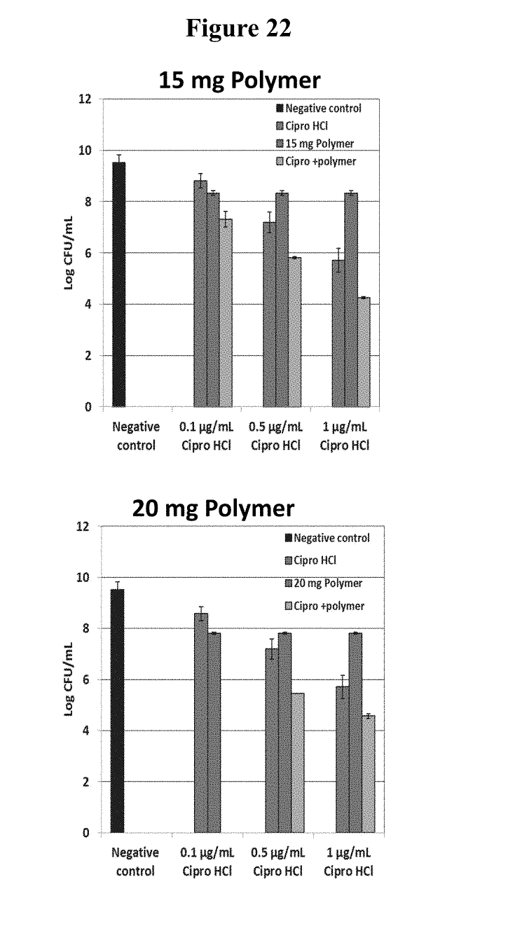

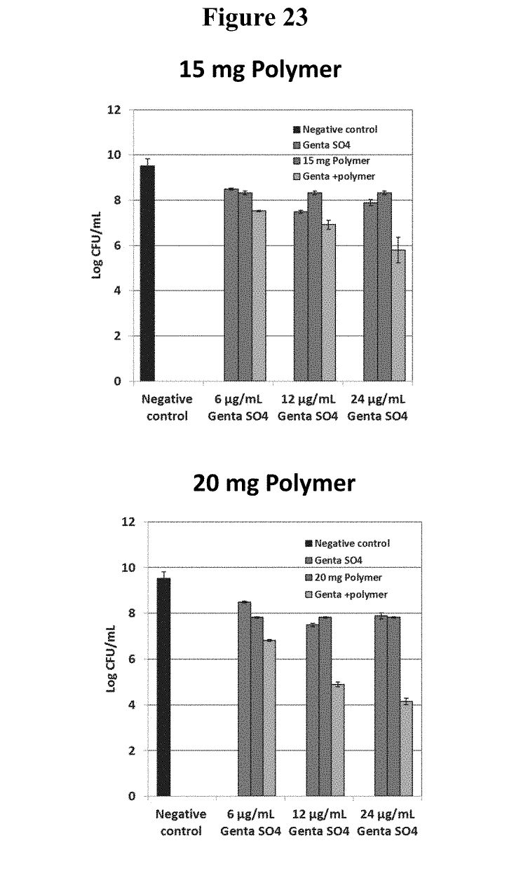

To investigate the effect of polymer needed as antibiotic adjuvant to inhibit P. aeruginosa growth, PAI-DHBA (20 mg) was tested in combination with different concentrations of ciprofloxacin and gentamycin. Bacterial growth significantly decreased in presence of the combined therapy compared to individual antibiotics at different concentrations (FIG. 22, 23).

### Example 9: Comparison to Traditional Iron Chelating Agents

One mL of M63 was used to culture the bacteria in 24 well clear flat bottom plates (Midsci). An overnight growth in LB was diluted to an OD₆₀₀=0.3. The diluted culture was then added to the M63 media for a starting OD₆₀₀=0.003. Test chelators including 20 mg PAI-DHBA, 208 mg of EDTA (equivalent to 500 μM) and 212 mg HBED (equivalent to 500 μM) were added directly to the media before bacterial inoculation. The CFU/mL was determined. The 24-well plate was wrapped with Parafilm to avoid evaporation. After incubating at 37° C. and 40 rpm for 12 h, entire wells were serially diluted in PBS (pH 7.4) and plated on LB agar by the drop plate method. The plates were incubated between 16-18 h at 37° C. CFU/mL was determined by counting single colonies after 5, 6 and 12 h of incubation.

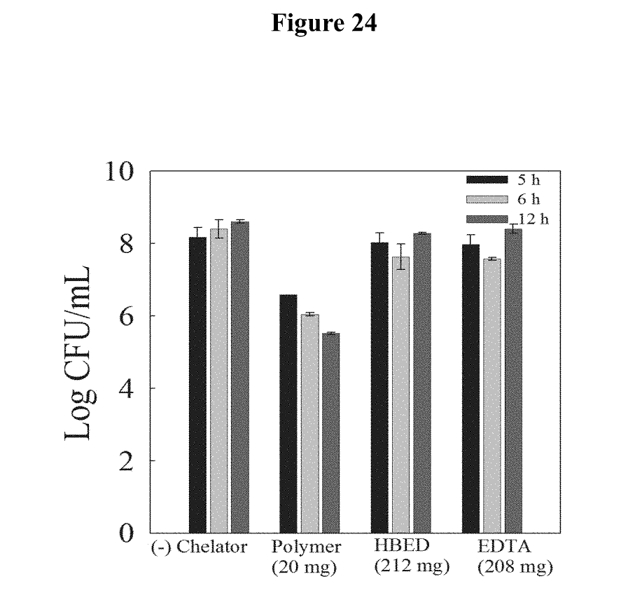

A small, transient difference between the control (no chelator), EDTA and HBED treatments was observed after 6 h of incubation but it disappeared after 12 h, with values resembling those of untreated media (FIG. 24). In contrast, sequestration of iron by PAI-DHBA efficiently depletes the medium from iron and inhibits bacterial growth.

### Example 10: Screening for Antimicrobial Activity of Polymers

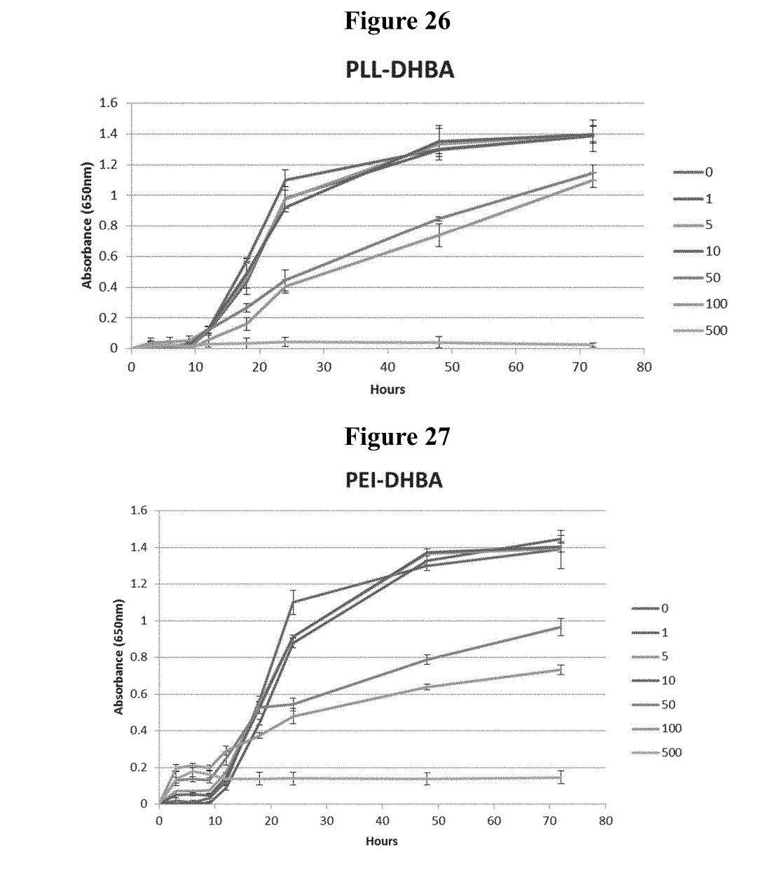

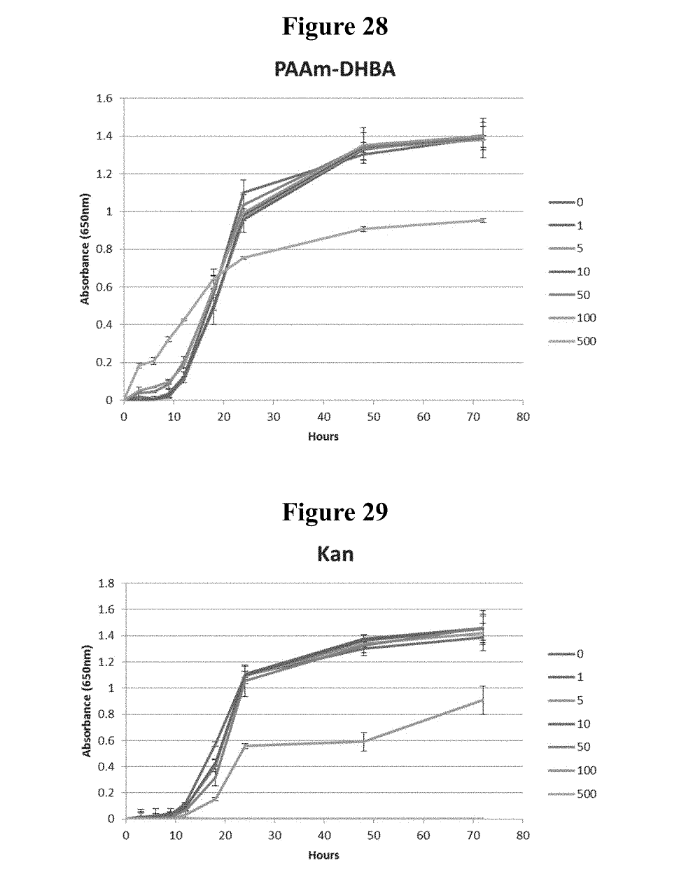

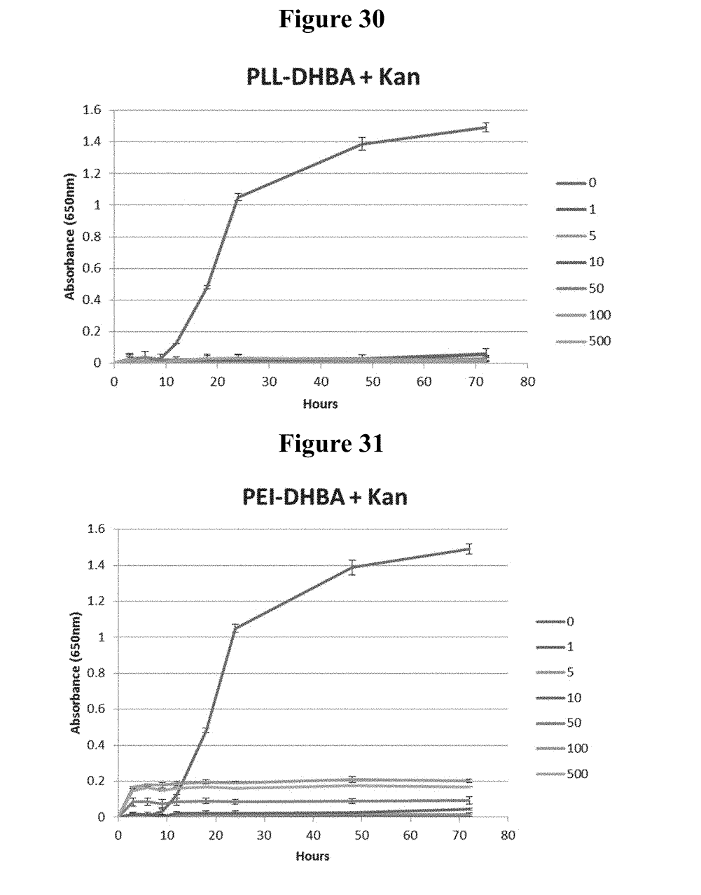

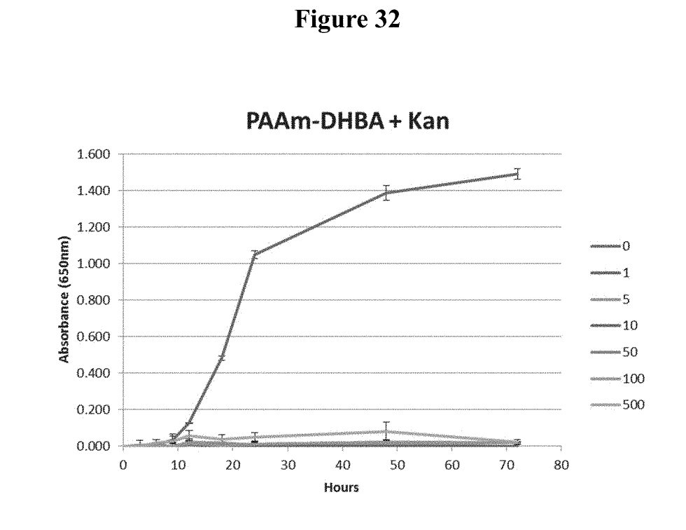

An overnight culture of B. pseudomallei MSHR305 was prepared and grown at 37 C, shaking. A 96 well non-treated plate was seeded with 50 uL of 2, 10, 20, 100, 200, 1000 μg/mL polymers or kanamycin, or a combination of polymer and kanamycin. The bacterial concentration of the overnight culture was determined and diluted to a uniform concentration. The bacteria were then added to the plate, bringing the final iron sequestrant polymer and kanamycin concentration to 1, 5, 10, 50, 100, 500 μg/mL, respectively. In the experiments involving the combination of kanamycin and polymer, each were provided at the stated final concentration (e.g., 1 μg/mL of polymer and 1 μg/mL of kanamycin). The structures of the sequestrant polymers tested are shown in FIG. 25. The plate was incubated at 37 C, stationary until the desired time-point. At that time, the absorbance was read (650 nm) to determine the overall growth curve. Bacterial growth was monitored at 0, 1, 3, 5, 8, 12, 18, 24, 48, and 72 hours post-inoculation. The average for the triplicate wells was ascertained, and the background subtracted to yield graphs showing absorbance change over time (FIGS. 26-32). As demonstrated in FIGS. 26-29, both kanamycin and iron sequestrant polymer were each effective separately to inhibit bacterial growth at higher concentrations. Referring now to FIGS. 30-32, a dramatic synergistic effect is achieved when kanamycin and iron sequestrant polymer are combined, even at the lowest concentrations of each; concentrations at which kanamycin and polymer alone were not effective in inhibiting bacterial growth.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method for treating a subject with or susceptible to a bacterial infection comprising:
administering a composition to the subject in an amount effective to treat or prevent the bacterial infection, wherein the composition comprises a plurality of polyamine polymer backbone chains and one or more chelators, wherein the one or more chelators are covalently coupled to one or more primary amines, respectively, of at least one of the plurality of polyamine polymer backbone chains through one or more amide bonds, respectively, wherein each of the one or more chelators has a benzene ring with more than one hydroxyl group at any position that is free.

2. The method of claim 1, wherein the composition is administered to the subject at a site harboring the bacterial infection.

3. The method of claim 2, wherein the administering step is by topical application of the composition to the site.

4. The method of claim 2, further comprising the step of administering an antibiotic agent to the site following administration of the composition.

5. The method of claim 2, wherein the composition further comprises an antibiotic agent.

6. The method of claim 2, wherein the site is an external wound on the subject.

7. The method of claim 2, wherein the site is a mucosal surface.

8. The method of claim 7, wherein the mucosal surface is bronchial, endometrial, gastric, penile, vaginal, olfactory, intestinal, anal, or oral.

9. The method of claim 1, wherein the bacterial infection is in the form of a biofilm.

10. The method of claim 1, wherein the bacterial infection is a gram-negative bacterial infection.

11. The method of claim 1, wherein the bacterial infection is a pseudomonas aeruginosa infection.

12. The method of claim 1, wherein the polyamine polymer backbone chains are polyallylamine or polylysine.

13. The method of claim 1, wherein the polyamine is selected from the group consisting of polyallylamine (PAA), polyvinyl formamide (PVF), polyvinylamide (PVA), polylysine (PLL), and polyethylenimine (PEI).

14. The method of claim 1, wherein the chelator is 2,3 dihydroxybenzoic acid or 2,3 dihydroxybenzaldehyde.

15. The method of claim 1, wherein the chelator is 2,3 dihydroxybenzaldehyde and the polyamine is polyallylamine.

16. The method of claim 1, further comprising the step of administering an antibiotic agent to the subject.

17. The method of claim 1, wherein the composition further comprises an antibiotic.

18. The method of claim 1, wherein the composition is a hydrogel.

19. The method of claim 1, wherein the molar ratio of A to B is from about 0.07 to about 0.17, wherein A is chelators covalently coupled to primary amines, and wherein B is total amines of the composition.

20. The method of claim 1, wherein the molar ratio of A to B is from about 0.03 to about 0.22, wherein A is chelators covalently coupled to primary amines, and wherein B is total amines of the composition.
